(19) United States
(12) Patent Application Publication
(10) Pub. No.: US 2022/0142559 A1
(43) Pub. Date: May 12, 2022

(54) Diagnosis Support Apparatus and Diagnosis Support Program

(71) Applicant: STAR MEDICAL, Inc., Tokyo (JP)

(72) Inventor: Satoru Kawada, Tokyo (JP)

(21) Appl. No.: 17/604,959

(22) PCT Filed: Apr. 21, 2020
(86) PCT No.: PCT/JP2020/017184

§ 371 Date: Oct. 19, 2021

(30) Foreign Application Priority Data
Apr. 25, 2019 (JP) 2019-084716

(51) Int. Cl. A61B 5/00; G16H 30/40

(52) U.S. Cl. CPC A61B 5/4233; G16H 30/40; A61B 2562/0247; A61B 5/7435; A61B 5/7425

(57) ABSTRACT

A diagnosis support apparatus includes an examination unit, a landmark setting unit, an analysis-and-diagnosis unit, and a display control unit. The examination unit acquires a pressure when an object passes through a gastrointestinal tract inside a subject from a sensor placed along a longitudinal direction of the gastrointestinal tract. The landmark setting unit automatically sets a landmark according to a predetermined condition based on an examination result acquired by the examination unit. The analysis-and-diagnosis unit analyzes the examination result using the set landmark to acquire an analysis result. The display control unit displays the analysis result on a display unit.

### TECHNICAL FIELD

Embodiments of the present invention relate to a diagnosis support apparatus and a diagnosis support program.

### BACKGROUND

In the human gastrointestinal tract, objects in the gastrointestinal tract move by repeatedly contracting and relaxing the gastrointestinal tract wall. For example, in a case where the gastrointestinal tract is the esophagus, the swallowed food and drink are moved from the pharynx to the stomach by sequentially performing a peristaltic movement (muscular contraction) of the esophagus.

The peristaltic movement can also be seen from the fact that when the peristaltic movement of the esophagus is measured and represented by a waveform, peaks indicating a high pressure in the waveform move sequentially. The movement leads to an abnormal pressure if a person has some disease, and therefore, it is necessary to grasp the presence or absence, location, and the like of the disease based on a pressure value.

Here, various methods have been developed so far for an examination for measuring a pressure in accordance with the contraction of the esophagus, performed to diagnose the esophageal motility disorder. Examples of the method may include an infused catheter method and a transducer method.

The infused catheter method is a method of flowing water continuously and slowly in a catheter having a plurality of side holes to measure a pressure that impedes the flow of water from the side holes. In the infused catheter method, a pressure measurement sensor is attached to a pump for making the water flow. On the other hand, the transducer method is a method of measuring an internal pressure of the esophagus with the pressure measurement sensor directly attached to a catheter and inserted into the esophagus. In addition, in such methods, there is the small number of side holes or pressure measurement sensors provided in the catheter.

Here, in order to diagnose the esophageal motility disorder, for example, it is important to evaluate a function of the lower esophageal sphincter (LES) between the esophagus and the stomach. However, the above-described methods have caused inconveniences such as the inability to continuously measure the LES and the complicated measurement.

Therefore, a high-resolution manometry (HRM) has been developed as an examination method for eliminating these inconveniences. The method is an improved method of the transducer method described above. In the examination, the HRM utilizes a catheter in which, for example, 36 pressure measurement sensors are placed. By using such a catheter, it is possible to continuously measure the internal pressure over the entire esophagus from the pharynx to the stomach without a blind spot between the sensors caused by the small number of pressure measurement sensors.

Moreover, when the HRM is used as an examination method, it is also possible to display an image of a change in pressure with a change in colors (topography display), such as red (high-pressure region) and blue (low-pressure region), in addition to displaying the change in pressure at each site in the esophagus with waveforms.

In order to diagnose such esophageal motility disorder, a plurality of times of examination is performed to improve the accuracy of the diagnosis. Therefore, it is necessary to always place the sensor that measures an item, for example, the LES or the upper esophageal sphincter (UES), required for analysis (hereinafter, such an item is appropriately represented as a “landmark”), at the same position in the esophagus regardless of the number of examinations.

However, when the HRM examination method is used, it is conceivable that the catheter moves at each measurement due to a liquid that a subject drinks at each examination or the contraction of the esophagus. That is, the position of the sensor in the esophagus may be changed for each time of measurement. Therefore, indeed, a doctor who makes the diagnosis adjusts the position of the sensor, that is, the position of the landmark for each examination after the examination is completed.

### BRIEF SUMMARY

However, the above-described examination for diagnosing the esophageal motility disorder needs to be repeated 10 times, at least 8 times, for each subject, in general. Under such a state, if the doctor has to adjust the position of the landmark for each time of measurement, the procedure to the diagnosis becomes very complicated.

Moreover, in order to adjust the positions of the landmarks, adjustment processing needs to be performed by displaying a display image of each time of measurement to be adjusted. Therefore, when the adjustment is required for a plurality of examinations, an image should be displayed for each examination. Further, it is not possible to view the entire examination image at once, which is complicated and inconvenient.

The present invention has been made to solve the above problems, and an object of the present invention is to automatically set the landmark required for analysis of examination results in order to diagnose the esophageal motility disorder. Further, at the time of confirming the positions of the landmarks that are automatically set after the analysis, images for each time of measurement are listed.

The diagnosis support apparatus according to an aspect includes an examination unit, a landmark setting unit, an analysis-and-diagnosis unit, and a display control unit. The examination unit acquires a pressure when an object passes through a gastrointestinal tract inside a subject from a sensor placed along a longitudinal direction of the gastrointestinal tract. The landmark setting unit automatically sets a landmark according to a predetermined condition based on an examination result acquired by the examination unit. The analysis-and-diagnosis unit analyzes the examination result using the set landmark to acquire an analysis result. The display control unit displays the analysis result on a display unit.

A diagnosis support program according to an aspect causing a diagnosis support apparatus to execute a process including a step of acquiring an examination result for an examination that measures a pressure when an object passes through a gastrointestinal tract inside a subject, a step of receiving an analysis request from a doctor and starting analysis processing for the examination result, a step of automatically setting a landmark in each of a plurality of times of measurement constituting the examination result, and a step of analyzing the examination result using the set landmark and acquiring an analysis result.

Because such a configuration is adopted in the present invention, it is possible to automatically set the landmark required for analysis of the examination result in order to diagnose the esophageal motility disorder. Further, at the time of confirming the positions of the landmarks that are automatically set after the analysis, it is possible to list images for each time of measurement.

### BRIEF DESCRIPTION OF THE DRAWINGS

FIG. 1 is a block diagram illustrating an overall configuration of a diagnosis support apparatus according to an embodiment.

FIG. 2 is an explanatory diagram illustrating an example of a display mode of an analysis result, a diagnosis result, and a reason of the diagnosis result displayed on a display unit according to the embodiment.

FIG. 3 is a display example of a list of images showing examination result for each time of measurement in the examination, displayed on the display unit according to the embodiment.

FIG. 4 is an example of an enlarged view of the image showing the examination result for each time of measurement, displayed in the list of the images illustrated in FIG. 3.

FIG. 5 is a display example illustrating an image of the time of measurement to be corrected of a landmark, selected from the list of the images and displayed on the display unit according to the embodiment.

FIG. 6 is a flowchart illustrating a rough flow when diagnosing a gastrointestinal motility disorder according to the embodiment.

FIG. 7 is a flowchart illustrating a flow of the examination performed when diagnosing the gastrointestinal motility disorder according to the embodiment.

FIG. 8 is a flowchart illustrating a flow of the analysis and diagnosis performed when diagnosing the gastrointestinal motility disorder according to the embodiment.

FIG. 9 is a flowchart illustrating a flow of the analysis and diagnosis performed when diagnosing the gastrointestinal motility disorder according to the embodiment.

### DETAILED DESCRIPTION

Hereinafter, embodiments will be described in detail with reference to the drawings.

Configuration of Diagnosis Support Apparatus

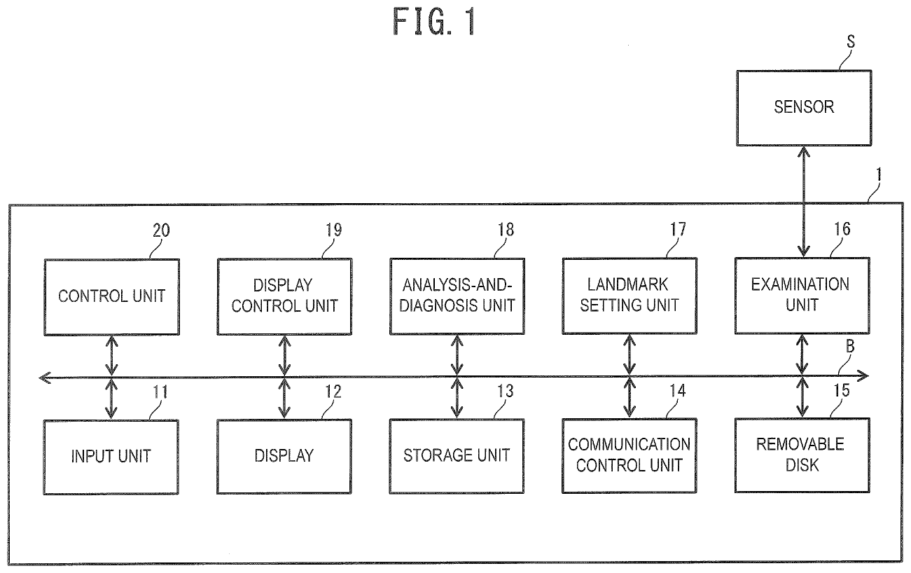

FIG. 1 is a block diagram illustrating an overall configuration of a diagnosis support apparatus 1 according to an embodiment. The diagnosis support apparatus 1 is, for example, a personal computer. In addition, the diagnosis support apparatus 1 may be, for example, a workstation capable of being connected to various network systems constructed in medical institutions. Further, the diagnosis support apparatus 1 may be a dedicated device for diagnosis support configured to execute various functions to be described below.

The diagnosis support apparatus 1 is a device used by an examiner or a doctor who examines and diagnoses the gastrointestinal motility disorder (hereinafter, these persons are collectively referred to as a “medical professional”), and is placed in a so-called examination room or consultation room. However, in addition to using the diagnosis support apparatus in a stationary manner, it is possible to adopt a form of a portable type or mobile type in consideration of use in a home visit examination or the like.

The medical professional uses the diagnosis support apparatus 1 and examines and diagnoses the gastrointestinal motility disorder. Here, the “gastrointestinal tract” corresponds to various parts, but in the following description, the esophagus will be taken as an example.

The diagnosis support apparatus 1 according to the embodiment includes an input unit 11, a display 12, a storage unit 13, a communication control unit 14, a removable disk 15, an examination unit 16, a landmark setting unit 17, an analysis-and-diagnosis unit 18, a display control unit 19, and a control unit 20. Further, each of these units is connected to each other via a bus B.

The input unit 11 accepts various input operations by medical professionals, such as selection of an examination protocol, image display, image switching, mode designation, or various settings. The operation of the medical professional input via the input unit 11 is converted into an input signal and transmitted to the control unit 20 or the like via the bus B.

As the input unit 11, for example, a graphical user interface (GUI) or an input device such as a button, a keyboard, a trackball, or a touch panel displayed on the display 12 is able to be used.

The display 12 displays processing results of the control unit 20 and the like. In addition, the display 12 displays examination conditions required for the diagnosis of the esophageal motility disorder, or displays an analysis result or automatic diagnosis result based on the examination result. Further, an output signal is received from the display control unit 19 via the bus B, and for example, when correction of landmark is required, a list of the examination results for each time of measurement is displayed.

The display 12 may use a liquid crystal display, an organic electroluminescence (EL) display, or the like. The display 12 constitutes a part of the display unit, including an external display device such as a printing machine connected to the diagnosis support apparatus 1 to be described below, in addition to the display 12.

In the embodiment of the present invention, the input unit 11 and the display 12 are described as one component of the diagnosis support apparatus 1 as illustrated in FIG. 1, but are not limited to such a configuration. For example, the display 12 may be configured separately from the diagnosis support apparatus 1, instead of a component of the diagnosis support apparatus 1. Moreover, the input unit 11 may be a touch panel using a separate display.

The storage unit 13 is composed of, for example, a semiconductor or a magnetic disk, and stores programs or data executed by the control unit 20 and the like. In addition, for example, the storage unit 13 stores a signal transmitted from the sensor S, for example, an examination result, when the above-described sensor S is connected to the diagnosis support apparatus 1. Alternatively, the storage unit 13 stores a reference value (threshold) or the like used when analysis or diagnosis processing is performed.

Furthermore, the storage unit 13 stores an examination program and a diagnosis support program used when diagnosing the esophageal motility disorder. Although the examination program and the diagnosis support program are shown as separate programs here, both programs may be configured as one program and stored in the storage unit 13.

In the embodiment of the present invention, the description will be described below on the premise that the storage unit 13 is provided in the diagnosis support apparatus 1. However, an external storage medium such as a server device or a hard disc drive, which is connected to the diagnosis support apparatus 1 wirelessly or by wire, may be used as the storage unit.

Here, it is premised that the above-described examination programs or the diagnosis support programs, the signals transmitted from the sensor S, and the like are all stored in the storage unit 13. However, it is also possible to provide a plurality of storage units 13 to separately store the examination program, the diagnosis support program, the signal transmitted from the sensor S, and the like.

The communication control unit 14 serves to connect a medical image diagnosis device (modality), a server device, a workstation, or the like (not illustrated) being connected to each other via a communication network (not illustrated) to the diagnosis support apparatus 1. Examples of a communication network N include networks such as local area network (LAN) and the Internet.

Moreover, the standard regarding information and medical images exchanged with other devices via the communication control unit 14 and the communication network may be any standard such as digital imaging and communication in medicine (DICOM). In addition, the connection with the communication network and the like is made wirelessly or by wire.

The removable disk 15 is an optical disk or a flexible disk, and the signal read and written by the disc drive is transmitted to and received from the control unit 20 via the bus B. It is also possible to use the removable disk 15 as a part of the storage unit 13 described above.

The examination unit 16 executes an examination for diagnosing the esophageal motility disorder. Here, the examination is performed by the HRM method described above. In the examination, a catheter (described below) is inserted through the nose of a subject to observe a state (flow) of objects (here, liquid) falling from the throat to the esophagus and the stomach by allowing the subject to swallow the liquid such as water. Specifically, a sensor provided in the catheter measures a pressure (internal pressure of the esophagus) when the liquid is drunk.

The examination itself becomes the base of the diagnosis by being performed, for example, 10 times (at least 8 times). In addition, the examination is not continuously performed plural times, but drinking of the liquid (start of measurement), completion of the measurement, and an interval are repeated. A waveform is acquired for each time of measurement. Then, analysis is performed based on the acquired waveform, and for example, the analysis result is represented by color-topography and displayed on the display 12. Further, it is possible to display the diagnosis result on the display 12 using the analysis result.

The sensor S is connected to the examination unit 16. For example, the sensor S is provided in a device called a catheter, which is different from the diagnosis support apparatus 1. When the examination of the esophageal motility disorder is performed, the catheter is inserted into the inside of the subject through the nose, thereby measuring a pressure at the time of contracting the esophagus in each site of the esophagus in which the sensor S is placed.

As described above, the sensor S is a pressure measurement sensor provided in the catheter and configured to measure the internal pressure of the esophagus. The form of the sensor does not matter so long as it is a pressure measurement sensor. The catheter includes, for example, 36 sensors S along a longitudinal direction of the catheter. Therefore, when the catheter is inserted into the esophagus, the sensors S are placed between an upper portion of the esophagus and a border between the esophagus and the stomach.

The specific processing of the examination unit 16 is generally as follows. That is, when the examination unit 16 receives, for example, a signal of executing the examination from the medical professional via the input unit 11, the examination unit 16 causes the display 12 to display an input screen of examination conditions and the like via the display control unit 19 and the like. Further, when the examination conditions and the like are determined and the examination is actually started, information (waveform data) regarding the internal pressure of the esophagus measured by the sensor S is acquired as an examination result. For example, the acquired examination result is transmitted to and stored in the storage unit 13.

In the examination required for diagnosing the esophageal motility disorder, about 10 measurements are performed as described above. The examination unit 16 transmits and stores the examination result for each time of measurement to and in the storage unit 13. However, the examination result of a plurality of times of measurement may be collectively transmitted to the storage unit 13.

When the examination is performed, for example, the peristaltic movement of the esophagus (muscular contraction) is sequentially performed from the pharynx to the stomach in a case of the healthy person. It can be seen from the fact that peaks indicating a high pressure sequentially move along a time axis based on the waveform data acquired by the sensor S. The movement may lead to an abnormal pressure if a person has some disease. In other words, the presence or absence, location, and the like of the disease are grasped based on a pressure value acquired by the examination.

That is, by performing the examination using the HRM examination method, it is possible to easily and appropriately evaluate a function of the esophageal motility, continuously from the upper esophageal sphincter (UES) described above to the lower esophageal sphincter (LES).

The landmark setting unit 17 automatically assigns a landmark to the examination result for each time of measurement when the medical professional requests analysis and diagnosis of the examination result. Here, the landmark is an item required for analyzing and diagnosing the examination result.

For example, it is possible for the landmarks to include swallow, GASTRIC, and the like, in addition to the UES and LES described above. First, a swallow (SW) represents a timing of swallowing at the time of examination. In addition, “GASTRIC” represents a position of the stomach.

As described above, when performing an examination, the sensor S provided in the catheter measures the internal pressure of the esophagus by inserting the catheter through the nose of the subject. However, the catheter may move vertically at each measurement because of a case where the liquid is swallowed for the examination, or contraction of the esophagus.

Moving the position of the catheter vertically means that the measurement position for each measurement is changed just as moving the position of the sensor S vertically, such that the landmark is also moved. Thus, it is difficult to obtain an accurate examination result.

Therefore, the doctor has set the positions of the landmarks of every time of measurement in the related art. That is, the doctor has set a landmark after all the measurements were completed or each time when the measurement was completed so as to eliminate the variation of the analysis result due to the movement of the catheter. However, it requires much time to set the landmark for each time of measurement, which may be inappropriate in terms of the diagnosis result obtained quickly without an error.

Therefore, the landmark setting unit 17 according to the embodiment of the present invention automatically sets a landmark for each time of measurement for the examination result obtained by completing the measurement. Specifically, first, the landmark setting unit 17 acquires the waveform data stored in the storage unit 13. Then, one landmark is set for each time of measurement.

For example, for the UES, the landmark setting unit 17 calculates an average waveform by obtaining a longitudinal waveform in a stationary state at, for example, an upper ⅓ portion of the waveform data. Then, a peak position exceeding a predetermined value is calculated from the calculated average waveform. When only one peak position is able to be calculated, the position is set as a UES position. In addition to this, when there is the peak position, an appropriate position is set as the UES position depending on whether the peak position is singular or plural.

For example, for the LES, the landmark setting unit 17 calculates an average waveform by obtaining a longitudinal waveform in a stationary state at, for example, a lower ⅓ portion of the waveform data. Then, a peak position exceeding a predetermined value is calculated from the calculated average waveform. When only one peak position is able to be calculated, the position is set as an LES position. In addition to this, when there is the peak position, an appropriate position is set as the LES position depending on whether the peak position is singular or plural.

In a case of the swallow, the landmark setting unit 17 grasps a pressure value in order based on the waveform of the UES, determines, for example, a point in time at which the pressure value drops as a swallow position, and sets a landmark of the swallow.

As such, the landmark setting unit 17 automatically sets the landmark for the examination result in all the times of measurement, such that it is possible to suppress the error as compared with a case where the doctor individually sets the landmark. Therefore, it is possible to eliminate the habits or the like of the doctor who sets the landmark and to quickly provide more common analysis result and diagnosis result.

The landmark set by the landmark setting unit 17 has been described above using some landmarks by way of example. The landmark setting unit 17 automatically sets one landmark for all the times of measurement, even for a landmark other than the above-described landmarks necessary for setting.

On the other hand, the analysis result or the diagnosis result is given for each time of measurement based on the landmarks that are automatically set by the landmark setting unit 17. Thereafter, the doctor checks the landmark, and in a case where the landmark is required to be corrected, the doctor individually corrects the landmark. In this case, the doctor manually resets the landmark, and in this case, the landmark setting unit 17 moves a position of the landmark according to an input work of the doctor via the input unit 11.

The analysis-and-diagnosis unit 18 calculates an analysis result and a diagnosis result based on the examination result when the doctor requests the result of the examination. In the following, calculating a result in all the times of measurement is represented as “analysis”, and calculating of information capable of reference when the doctor finally diagnoses based on the analysis result in all the times of measurement is represented as “diagnosis”.

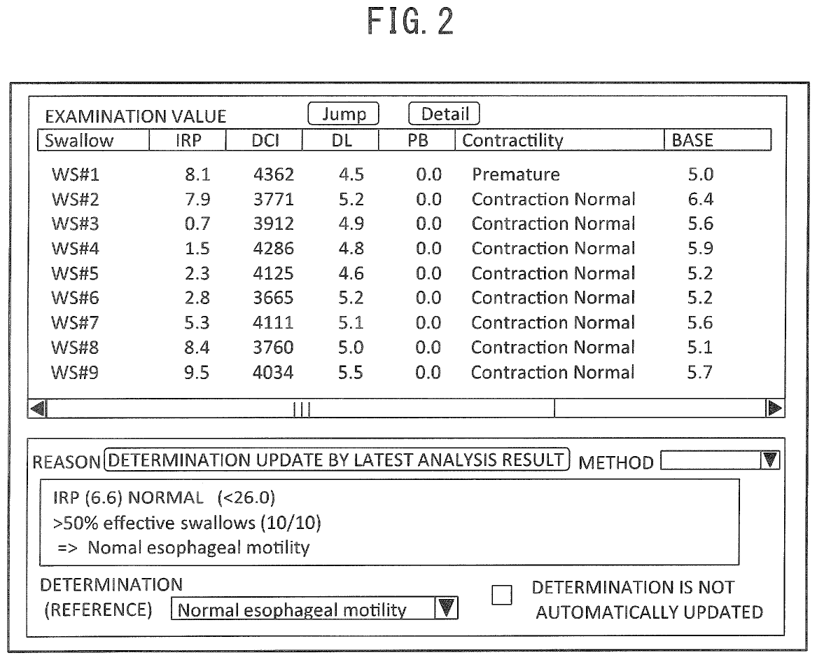

FIG. 2 is an explanatory diagram illustrating an example of a display mode of an analysis result, a diagnosis result, and a reason of the diagnosis result displayed on a display unit according to the embodiment. Specifically, FIG. 2 illustrates a state displayed on the display 12.

In the explanatory diagram in FIG. 2, a display area described as “examination value” on the upper left side and a display area described as “reason” on the upper left side are displayed in two rows above and below in a large frame. The analysis result is displayed in the display area of “examination value”, whereas the diagnosis result is displayed in the display area of “reason” together with the reason.

First, the analysis result for each time of measurement is shown in the display area of “examination value”. In the display area, 7 items of “Swallow”, “IRP”, “DCI”, “DL”, “PB”, “Contractility”, and “BASE” are shown from the left.

Among the items, “Swallow” refers to the time of measurement. Here, 10 items from “WS #1” to “WS #10” are displayed vertically, and each of “WS #1” to “WS #10” refers to the time of measurement. For example, “WS #1” refers to a first measurement. In addition, display of 10 items refers to that the examination is performed 10 times.

Therefore, the 5 items other than “Swallow” are the analysis results, and it is possible to grasp the analysis result for each item in each time of measurement while looking into the values horizontally for each time of measurement.

“IRP” refers to an abbreviation for “integrated relaxation pressure”, that is, an average pressure for four seconds (within a predetermined time) from the lower side at a portion of the LES, for example. Further, “DCI” refers to an abbreviation for “distal contractile integral”. “DL” refers to an abbreviation for “distal latency”, and “PB” is an abbreviation for “peristaltic breaks”. In addition, “Contractility” refers to contraction. Further, “BASE” is an average pressure of the esophageal body indicating a portion between the UES and the LES.

For example, when looking into the column of “WS #2”, the value of “IRP” is “7.9”, the value of “DCI” is “3771”, the value of “DL” is “5.2”, and the value of “PB” is “0.0”, and the item of “Contractility” is labeled as “Contraction”. In addition, the value of “BASE” is “6.4”. That is, the values of “IRP”, “DCI”, “DL”, “PB”, and “BASE” in the second examination are as described above, and the result of “Contractility” is normal.

It is possible to preset or arbitrarily set the item calculated as the analysis result, such as “IRP”. In addition, it is possible to arbitrarily select the item to be displayed on the display 12.

Moreover, two buttons “Jump” and “Detail” are provided on an upper portion of the analysis result of each time of measurement. When the time of measurement is selected and the “Jump” button is pushed down, the topography showing the examination result of the time of measurement is displayed on the display 12. On the other hand, for example, when a specific time of measurement is selected, and then the “Detail” button is pushed down, detailed information regarding the time of measurement including other items that are not displayed on the display 12 is displayed.

Next, the diagnosis result, which is determined based on the analysis result for all the times of measurement and capable of reference when the doctor diagnosis, is displayed in the display area of “reason”. In a screen example illustrated in FIG. 2, the diagnosis result is labeled as “determination (reference)”. In addition, the reason why it is labeled as “(reference)” herein is because the final diagnosis is obviously made by doctor, and the diagnosis support apparatus 1 merely provides reference information based on the analysis result.

In an upper part of the display area of “reason”, regarding the diagnosis result (determination) calculated based on the analysis result in all the times of measurement, how the diagnosis result (determination) is reached is briefly shown. Further, in the lower part of the display area of “reason”, “Normal esophageal mobility” is labeled as “determination (reference)”. “Normal esophageal motility” indicates that the function of the esophageal motility is normal, that is, not abnormal.

In addition, the display area is provided with a button of “determination update by latest analysis result” next to the display of “reason”. The button is a button for calculating the analysis result again when the doctor has a doubt of the analysis result or the determination result confirming the analysis result of each time of measurement, and as a result, setting the landmark manually again.

Moreover, a display area written as “method” is provided next to the “determination update by latest analysis result” button, and the display method is selectable. Further, a check box of “determination is not automatically updated” is provided below “determination (reference)”. If the check box is checked, it is possible to select processing that the determination is not automatically updated even when the analysis result is changed.

When the analysis-and-diagnosis unit 18 calculates the analysis result for each time of measurement, for example, the analysis-and-diagnosis unit 18 compares a reference value (threshold) for each item stored in the storage unit 13 with the obtained examination result. For example, in a case of the DCI, if a value of the examination result is compared with the reference value and the value of the examination result is less than the reference value, it is possible to derive a possibility that a person has some disease. That is, in a case of a healthy person in general, when food and drink are swallowed, the value of the DCI is within a certain range including the reference value. This is because the peristaltic movement is performed to send the swallowed food and drink from the esophagus to the stomach.

The reference value is, for example, a numerical value of a case or the like acquired so far, and is stored in the storage unit 13 in advance. It is also possible to combine and use a plurality of reference values when performing analysis.

Moreover, as described above, the analysis-and-diagnosis unit 18 analyzes the examination result based on the landmark set for each time of measurement. Further, the diagnosis result is calculated based on the analysis result in all the times of measurement. However, the analysis result used in calculation of the diagnosis result may use a numerical value appearing on the analysis result of each time of measurement such as the IRP as it is, or take and use an average value or median value. In addition, depending on the item, for example, the number of times, which is regarded as an abnormal value, is considered in comparison with the reference value. Furthermore, information as a base for calculating the diagnosis result is not limited to the analysis result.

For example, a phenomenon of pressure increase may be found in the analysis result. The phenomenon of increase in measured pressure in the esophagus is a phenomenon in which the pressure of the part or entire esophagus temporarily increases. That is, for example, it is a phenomenon in which the pressure increases because an entrance of the stomach, which is a destination of food and drink moving through the esophagus, does not open. Therefore, if this phenomenon of pressurization is found, it is presumed that a certain part of the esophagus has stenosis or the like.

Further, when looking into the pressure of BASE, for example, when the pressure tends to increase gradually, it is presumed that a certain part of the esophagus has stenosis or the like.

In the diagnosis support apparatus 1 according to the embodiment of the present invention, when the analysis-and-diagnosis unit 18 calculates the diagnosis result, the analysis-and-diagnosis unit 18 confirms whether or not the phenomenon of pressure increase occurs. The analysis-and-diagnosis unit 18 calculates the diagnosis result in consideration of whether or not a phenomenon such as contraction or peristalsis has occurred, in addition to the phenomenon of pressure increase.

Although the screen example illustrated in FIG. 2 illustrates an example of a state displayed on the display 12 as described above, the screen example may be displayed on the entire screen of the display 12, or for example, the screen example may be displayed on a small screen above the list showing the examination result for each time of measurement so that the screen example overlaps with the list.

The display control unit 19 controls the display unit including the display 12 and a printing machine to display, for example, the analysis result or the diagnosis result. Moreover, when the doctor sets the landmark again from the analysis result or the diagnosis result, the display control unit 19 performs control to display an image showing the examination result for each time of measurement on the display 12.

As described above, the analysis-and-diagnosis unit 18 analyzes and diagnoses the examination result based on the landmark automatically set by the landmark setting unit 17, and the results of the analysis and diagnosis are displayed on the display 12. However, when the doctor checks the analysis result, there may be doubts about the analysis result. In this case, the doctor often wants to check and refer to the examination result for each time of measurement as to why such an analysis result is derived.

When the display control unit 19 receives such a request from the doctor, the display control unit 19 displays a list of examination results for each time of measurement on the display 12. A list of the examination results in not only the time of measurement that the doctor especially wants to check, but also all the times of measurement of a subject to be diagnosed is displayed, such that the doctor can easily check the examination results.

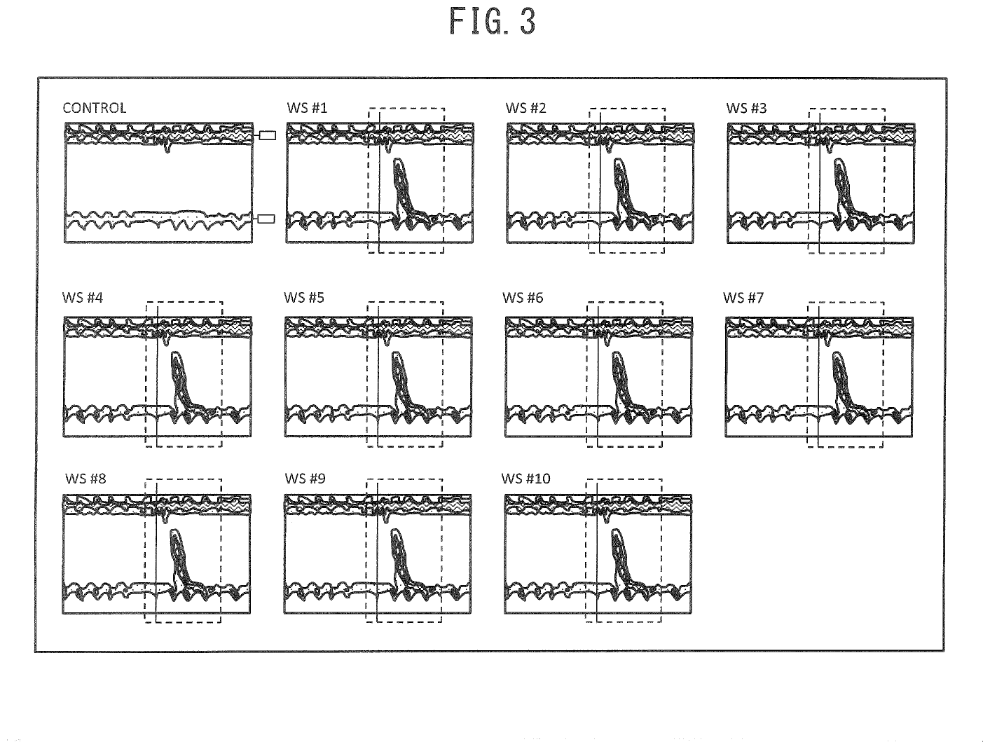

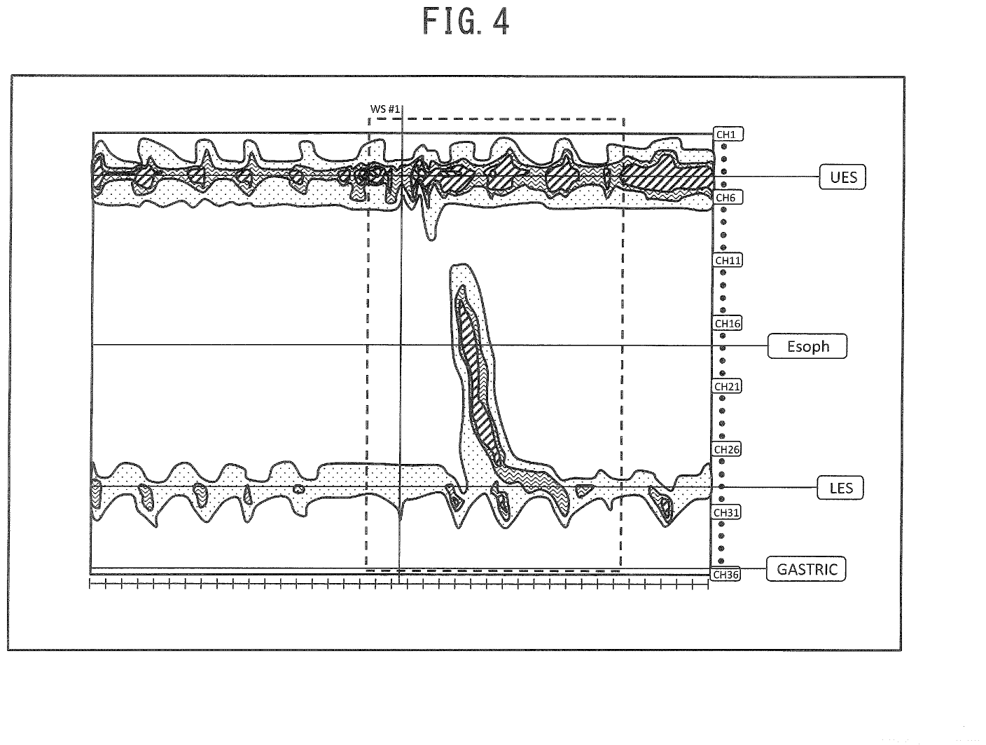

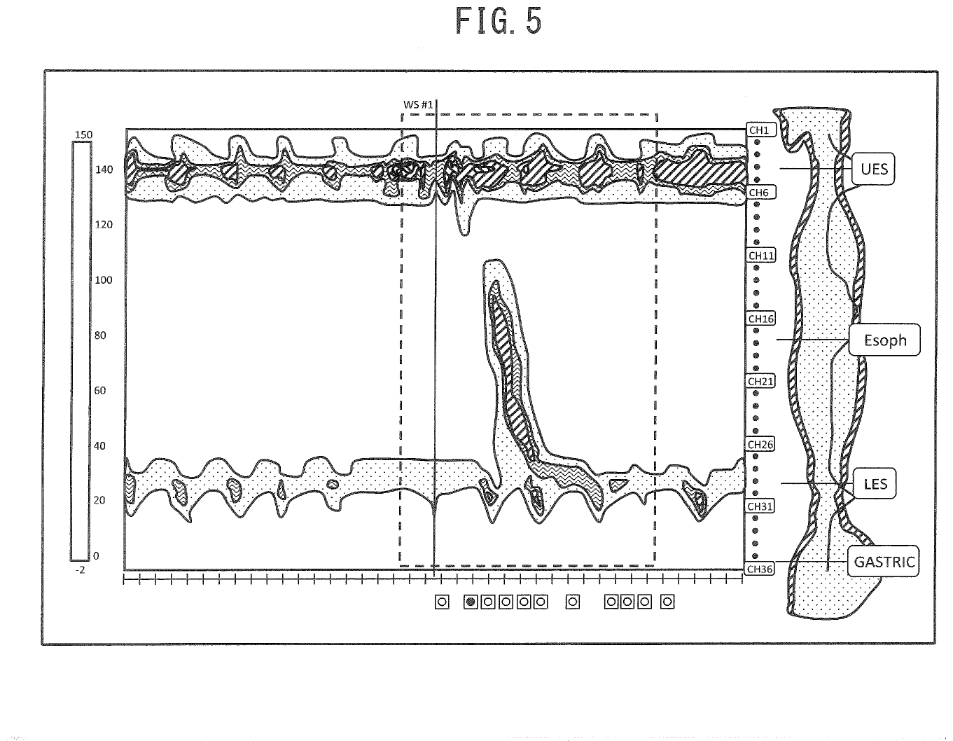

FIG. 3 is a display example of a list of images showing examination results for each time of measurement in the examination, displayed on the display unit according to the embodiment. The images of the examination results listed are so-called color-topography. Accordingly, the measured pressure in the esophagus is displayed in different colors for each pressure value. However, in FIG. 3 (and FIGS. 4 and 5 to be described below), color coding is represented by drawing with hatching instead of displaying in color.

Further, an area surrounded by a broken line is shown in the image for each time of measurement. The area indicated by the broken line shows an analysis range. The analysis range is shown by a range represented by a temporal width that includes before and after the time at which the subject swallows a liquid. In addition, the analysis range is able to be set as, for example, one condition in which the doctor analyzes and diagnoses before the examination.

Furthermore, a vertical line is shown in the broken line indicating the analysis range. The vertical line is defined as SW and refers to a start timing of swallowing by the subject as described above. The landmark setting unit 17 detects a portion where the UES is interrupted and determines a setting position of the vertical line.

The examination results of all 10 times of measurement from WS #1 to WS #10 are displayed in a display example illustrated in FIG. 3. In addition, a screen labeled as “CONTROL” is also displayed on the upper left of the screen. The “CONTROL” refers to a result obtained by measuring the pressure in the esophagus in a state in which the liquid is not swallowed, before starting the examination performed by swallowing the liquid.

In the image of “CONTROL”, because the subject does not swallow anything, it is shown that the pressure in the esophagus does not change. Accordingly, it is shown that the pressure in the middle portion of the esophagus does not change, unlike a case where the liquid is actually swallowed, following WS #1.

On the other hand, the images showing the examination results of each time of measurement from WS #1 to WS #10 other than “CONTROL” show the pressure in the esophagus when the subject swallows the liquid, respectively.

In FIG. 3, a list of 11 images of three images in height and four images in width including the image of “CONTROL” is displayed by the processing of the display control unit 19. However, it is possible to arbitrarily set the number of images displayed vertically and horizontally so long as the images are listed.

Further, the landmark in each image that is automatically set by the landmark setting unit 17 is not illustrated in FIG. 3. In the actual list, the landmark set by the landmark setting unit 17 based on the examination result for each of the times of measurement is also displayed.

FIG. 4 is an example of an enlarged view of the image showing the examination result for each time of measurement, displayed in the list of the images illustrated in FIG. 3. That is, FIG. 4 illustrates, in an enlarged manner, the image showing the examination result of WS #1 that is listed in FIG. 3.

As is clear from FIG. 4, an area represented by the color-topography has different hatching for each measured pressure, which refers to a pressure from a high pressure to a low pressure in the esophagus. In the color-topography illustrated in FIGS. 3 to 5 to be described below, hatching of diagonal lines extending from the upper left to the lower right, diagonal lines extending from the upper right to the lower left, wavy lines, and polka dots are added in descending order of pressure.

An area indicating a high pressure in the esophagus is shown to be continuous from the upper left to the lower right over time. The area shows that muscular contraction of the esophageal body as a clearance of bolus, which is in accordance with the gradual movement of the liquid swallowed by the subject from the upper part to the lower part in the esophagus, moves from the upper part to the lower part.

As described above, the area set as the analysis range is surrounded by the broken line. In addition, the vertical line indicating the start timing of swallowing by the subject is within the range surrounded by the broken line.

Labels with “CH1” to “CH36” displayed from the top to the bottom are shown in the right side of the area represented by color-topography. The labels refer to positions of the sensors S provided in the catheter, respectively. Here, because the labels with “CH1” to “CH36” are displayed, it can be seen that the catheter is provided with 36 sensors S (36 channels).

Although the number of channels is illustrated in FIG. 4, for example, a length from the upper part to the lower part of the esophagus may be displayed in units of, for example, cm. Moreover, it is also possible for the number of channels not to be displayed in the first place.

In addition, a scale referring to a time axis is displayed below the area represented by color-topography, which refers to elapse of time from left to right.

A landmark is shown next to the labels of the number of channels. The landmark is automatically set by the landmark setting unit 17, as described above. In FIG. 4, four landmarks, “UES”, “Esoph”, “LES”, and “GASTRIC”, are illustrated. Here, “Esoph” is a landmark indicating the almost central portion of the esophagus in length.

FIG. 5 is a display example illustrating an image of the time of measurement to be corrected of a landmark selected from the list of the images and displayed on the display unit according to the embodiment. The display example is displayed on the display 12 by the display control unit 19 when the doctor recognizes that the landmark in the examination result needs to be corrected and selects the examination result from a list of the examination results for each time of measurement displayed.

As can be seen from the display example illustrated in FIG. 5, a content displayed by color-topography is, for example, as described in FIG. 4. Furthermore, a color bar showing the pressure is displayed on the left side of the display of the color-topography in the display example of FIG. 5. Although the color coding is not illustrated in FIG. 5, the color coding that indicates pressures, for example, red indicating a high pressure and blue indicating a low pressure, is illustrated, as described above. In addition, a number (unit: mmHg) indicating the pressure in the esophagus is illustrated along the color bar.

On the other hand, a model diagram illustrating a state in which the esophagus is cut in the longitudinal direction is displayed on the right side of the color-topography. In addition, the pressure at each position superimposed on the model diagram is illustrated as a graph.

The landmarks described in FIG. 4 are displayed on the right side of the color-topography so as to be superimposed on the model diagram. For example, the doctor operates various devices constituting the input unit 11, specifically, for example, with a mouse, grabs a landmark to be corrected to move vertically, and is thus able to arrange the landmark at a position where he/she thinks appropriate.

A label indicating the time of measurement is provided below the color-topography, that is, further below the scale of the time axis. As illustrated in FIG. 3, there are 11 examination results listed including “CONTROL”. Then, the time of measurement currently displayed is marked with a black circle. The color-topography illustrated in FIG. 5 is WS #1, and the black circle also indicates that the displayed color-topography is WS #1.

The reason why the second display from the left is marked with the black circle even though the color-topography of WS #1 is displayed is that the leftmost display indicates the color-topography of “CONTROL”.

The control unit 20 collectively controls each unit of the diagnosis support apparatus 1. The control unit 20 receives, for example, an operation instruction from the medical professional via the input unit 11 as an input signal, and controls each unit so that a desired operation is performed.

The control unit 20 includes, for example, a central processing unit (CPU), read-only memory (ROM), and random-access memory (RAM), which are not illustrated.

The CPU reads and executes a boot program for starting the diagnosis support apparatus 1 from the ROM based on the input signal from the input unit 11, and reads various operating systems stored in the storage unit 13. In addition, the CPU controls various devices based on the input signal from other external devices (not illustrated in FIG. 1) via the input unit 11 and the like. Furthermore, the CPU is a processing device that reads the program and data stored in the RAM, the storage unit 13, or the like and loads the program and data into the RAM, and also implements a series of processing such as data calculation and processing based on a command of the program read from the RAM.

In the embodiment of the present invention, the description has been made on the premise that the “display control unit 19” and the “control unit 20” are distinguished as described above, but the control unit 20 may be configured to execute the processing of the display control unit 19 as described above.

That is, the control unit 20 may be provided with not only the function of the display control unit 19, but also functions of each of the examination unit 16, the landmark setting unit 17, and the analysis-and-diagnosis unit 18, as an “examination function”, a “landmark setting function”, and an “analysis-and-diagnosis function”, respectively.

Operations of each of the examination unit 16, the landmark setting unit 17, the analysis-and-diagnosis unit 18, and the display control unit 19 or operations when the control unit 20 has the functions of each of the units are based on the premise of causing a processor to execute a diagnosis support program stored in, for example, the storage unit 13.

The term “processor” as used herein refers to a circuit such as, for example, a dedicated or general central processing unit (CPU) arithmetic circuit (circuitry), an application specific integrated circuit (ASIC), or a programmable logic device (for example, a simple programmable logic device (SPLD), a complex programmable logic device (CPLD), and a field programmable gate array (FPGA)).

The processor implements the function by reading and executing a program stored in the storage unit 13 or directly incorporated in the circuit of the processor, for example. The storage unit for storing the program may be individually provided for each processor, or for example, may store a program corresponding to the function of the examination unit 16 or the like in FIG. 1, and furthermore, may adopt the configuration of the storage unit 13 illustrated in FIG. 1. For example, a storage device such as a general RAM such as a semiconductor or a magnetic disk or a hard disc drive (HDD) is applied to the configuration of the storage unit.

Furthermore, each function of the examination unit 16, the landmark setting unit 17, the analysis-and-diagnosis unit 18, and the display control unit 19 may be configured with the control unit 20 as a control circuit, and each of the examination unit 16, the landmark setting unit 17, the analysis-and-diagnosis unit 18, and the display control unit 19 may be individually configured as an examination circuit, a landmark setting circuit, an analysis-and-diagnosis circuit, or a display control circuit.

Operation

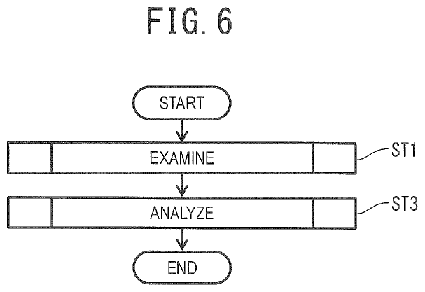

Next, processing in the diagnosis support apparatus 1 will be described with reference to FIGS. 6 to 9. FIG. 6 is a flowchart illustrating a rough flow when diagnosing a gastrointestinal motility disorder according to the embodiment. When the doctor diagnoses the esophageal motility disorder, it is possible to be roughly divided into an examination processing step (ST1) and an analysis-and-diagnosis processing step (ST3).

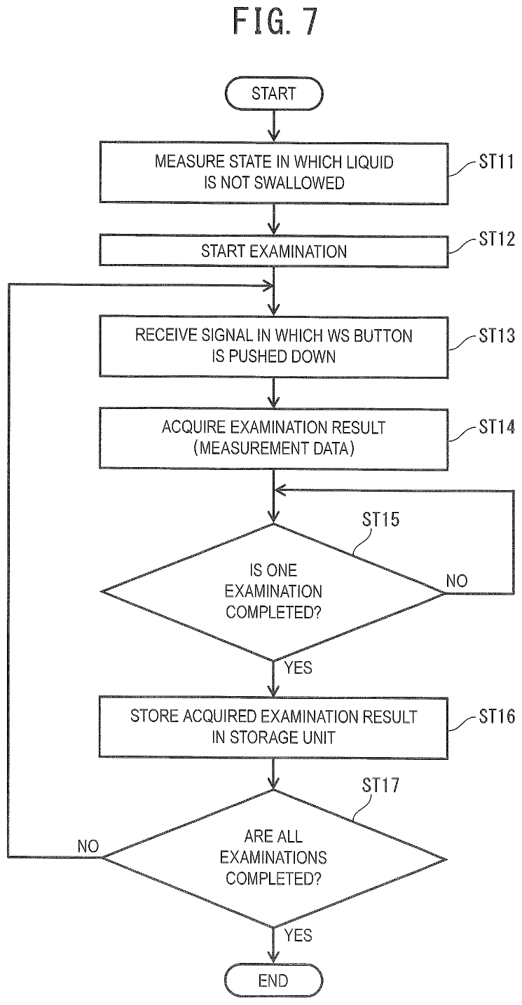

First, to describe the processing step of the analysis and diagnosis, the examination processing step will be described. FIG. 7 is a flowchart illustrating a flow of the examination performed when diagnosing the gastrointestinal motility disorder according to the embodiment.

The measurement is performed in a state in which the liquid is not swallowed before the subject actually swallows the liquid and the pressure in the esophagus is measured (ST11). ST11 is “CONTROL” described above. Because the liquid is not swallowed, the change in pressure in the esophagus is not measured as illustrated in FIG. 3. Although not illustrated in the flowchart, the measured result in the case of “CONTROL” is also transmitted from the examination unit 16 to the storage unit 13 as an examination result and stored.

Next, an examination for measuring the pressure in the esophagus in a state in which the subject swallows the liquid is started (ST12). At the timing when the subject swallows the liquid, the medical professional pushes down a swallow button, for example, which constitutes the input unit 11. The examination unit 16 receives a signal in which the swallow button is pushed down (ST13), and acquires an examination result (measurement data) showing the pressure in the esophagus acquired by the sensor S (ST14).

The examination unit 16 determines whether or not all measurement data has been transmitted from a plurality of sensors S provided along the longitudinal direction of the catheter (ST15), and stands by as it is when the transmitted data has not been transmitted from all the sensors S (NO in ST15). On the other hand, when it is determined that the transmitted data is transmitted from all the sensors S (YES in ST15), one examination is completed, and therefore, the acquired examination result is transmitted to and stored in the storage unit 13 (ST16).

As described above, the examination for diagnosing the esophageal motility disorder is performed a predetermined number of times, for example, 10 times. Then, the examination is continuously performed. Therefore, the examination unit 16 continuously stores the examination results for a predetermined number of times in the storage unit 13.

The examination unit 16 determines whether or not the examination results of 10 times of measurement from WS #1 as the first examination to WS #10 as the tenth examination are acquired, that is, all ten examinations are completed by confirming that the tenth examination is executed (ST17).

When the examination unit 16 does not receive the examination completion signal from the medical professional via the input unit 11 (NO in ST17), the examination processing step described above is executed again. On the other hand, when the examination unit 16 receives the examination completion signal from the medical professional via the input unit 11 (YES in ST17), the examination for the subject is completed.

The examination unit 16 is able to determine the examination completion, instead of receiving the examination completion signal from the medical professional via the input unit 11. In this case, when the examination unit 16 determines that all the examinations have not been completed, the examination processing step described so far is executed again. On the other hand, when the examination unit 16 determines that all the examinations have been completed, the examinations for the subject are completed.

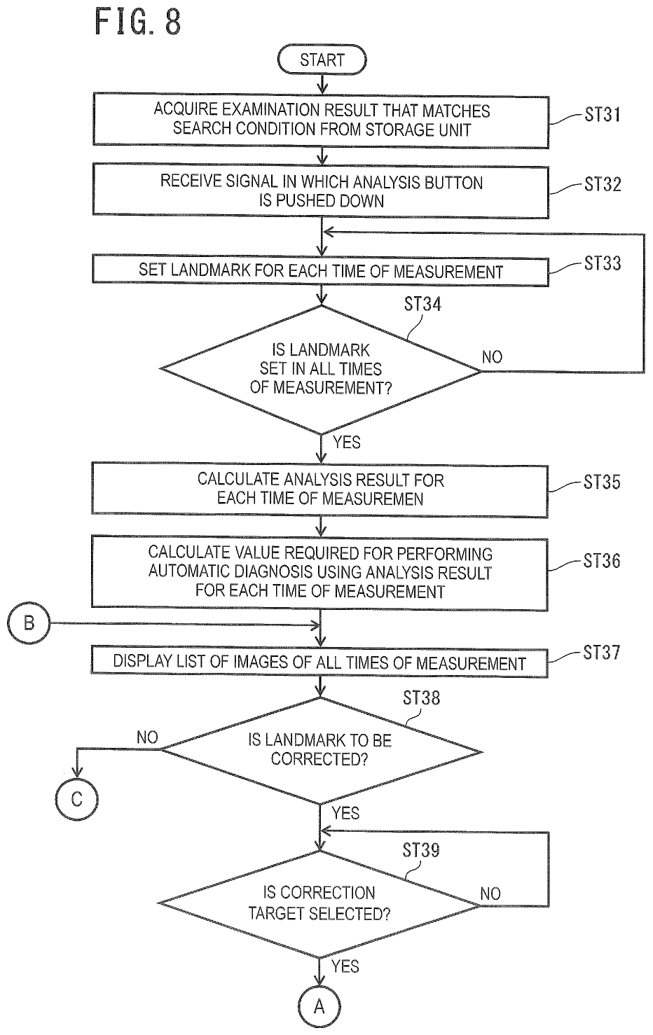

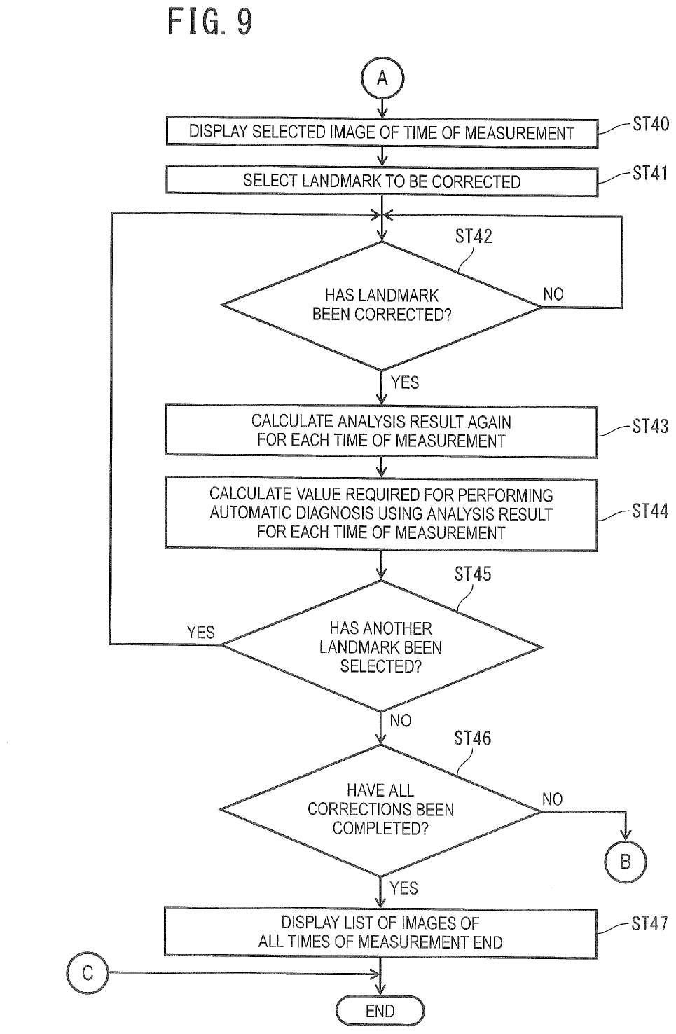

Next, the analysis-and-diagnosis processing step will be described with reference to FIGS. 8 and 9. FIGS. 8 and 9 are flowcharts illustrating a flow of the analysis and diagnosis performed when diagnosing the gastrointestinal motility disorder according to the embodiment.

When the doctor examines the subject, it is necessary to diagnose the condition of the subject based on the examination result. Therefore, the diagnosis support apparatus 1 provides an analysis result and a diagnosis result based on the examination result.

The doctor inputs, for example, an ID of the subject when the analysis-and-diagnosis processing step is started. An ID or the like of the subject is a search condition when the analysis-and-diagnosis unit 18 searches for the examination result. Further, for example, the analysis-and-diagnosis unit 18 may extract the search condition from the content displayed on the display 12 without inputting a special search condition by the doctor. The analysis-and-diagnosis unit 18 acquires the examination result that matches the search condition from the storage unit 13 (ST31).

Then, the doctor requests the diagnosis support apparatus 1 to present the analysis result and the diagnosis result based on the examination result (analysis request). Specifically, for example, the analysis-and-diagnosis unit 18 receives a signal in which an “All Calc” button (represented as an “analysis button” in FIG. 8) constituting the input unit 11 is pushed down to start the processing step for analysis and diagnosis (ST32).

The analysis-and-diagnosis unit 18 transmits the acquired examination result to the landmark setting unit 17, and the landmark setting unit 17 receives the examination result and sets the landmark for each time of measurement (ST33).

The landmark setting unit 17 determines whether or not the landmark has been set for the examination results of all the times of measurement (ST34), and if the landmark has not been set (NO in ST34), processing for setting the landmark is continuously performed for each time of measurement. On the other hand, when the landmark has been set for the examination results of all the times of measurement (YES in ST34), the information is transmitted to the analysis-and-diagnosis unit 18.

Here, the analysis-and-diagnosis unit 18 acquires the examination result from the storage unit 13 and passes the result to the landmark setting unit 17, but it does not have to be this process. That is, the landmark setting unit 17 receives the signal from the analysis button first. Then, the landmark setting unit 17 may perform processing of acquiring a necessary examination result from the storage unit 13, setting the landmark for each time of measurement, and transmitting the information to the analysis-and-diagnosis unit 18.

The analysis-and-diagnosis unit 18 calculates the analysis result for each time of measurement based on the examination result for each time of measurement at which the landmark is set (ST35). Furthermore, the analysis-and-diagnosis unit 18 calculates a value required for performing automatic diagnosis based on the analysis results for all the times of measurement (ST36).

Then, the display control unit 19 displays a list of images of all the times of measurement on the display 12 (ST37). This state is the display example illustrated in FIG. 3.

The doctor reviews the examination results of all the times of measurement listed on the display 12. Alternatively, the doctor is able to intensively check the worrisome examination result of the time of measurement, and further check the examination results of the times of measurement before and after the time of measurement.

The analysis-and-diagnosis unit 18 is also able to display the calculated analysis result, the diagnosis result, and a reason for deriving the diagnosis result on the display 12. The analysis results and the like displayed on the display 12 are as illustrated in FIG. 2.

As described above, the analysis-and-diagnosis unit 18 is able to display not only the list of all the times of measurement, but also the analysis result, the diagnosis result, and the reason for the diagnosis on the display 12 as appropriate, as information for supporting the diagnosis of the doctor. The doctor makes a diagnosis for the subject by referring to the information.

The doctor looks into the list of all the times of measurement, the analysis result, and the like, and checks the worrisome point. When the worrisome point is found, the doctor will further need to check the point. In other words, the checking work by the doctor leads to landmark correction processing. In the diagnosis support apparatus 1, it is confirmed whether or not any signal has been received via the input unit 11 for executing the landmark correction processing by the doctor. That is, it is determined whether or not the landmark is to be corrected (ST38).

As a specific processing, for example, the doctor pushes down the “Jump” button or “Detail” button illustrated in FIG. 2, and the display control unit 19 receives the signal (YES in ST38) and displays information required for the doctor to check the diagnosis on the display 12.

The display control unit 19 determines whether or not the doctor has selected an image to be corrected via the input unit 11 (ST39). When the correction target has not been selected yet (NO in ST39), the display control unit 19 continues to display a list of the images of all the times of measurement on the display 12.

On the other hand, when the correction target has been selected (YES in ST39), the display control unit 19 displays only the selected examination result of the time of measurement on the display 12 (ST40 in FIG. 9). The state is the display example illustrated in FIG. 5.

The doctor looks into the displayed examination result of the time of measurement and corrects the position of the landmark automatically set by the landmark setting unit 17. Specifically, as described above, the landmark is selected using the input unit 11 (ST41), the position of the landmark is moved vertically on the screen, and the landmark is set again at a position where the doctor thinks appropriate.

The display control unit 19 determines whether or not the doctor has reset the landmark and the correction has been completed (ST42). That is, the display control unit 19 determines that the correction of the landmark has not been completed while the doctor is executing the processing of moving the landmark (NO in ST42). On the other hand, for example, if the doctor releases the landmark that he/she grabbed to move the landmark or does not move the landmark for a certain period of time, the display control unit 19 determines that the landmark correction has been completed (YES in ST42).

The analysis-and-diagnosis unit 18 calculates the analysis result for each time of measurement again based on the examination result for each time of measurement at which the landmark is set (ST43). Furthermore, the analysis-and-diagnosis unit 18 calculates a value required for performing automatic diagnosis based on the analysis results for all the times of measurement (ST44).

Obviously, the landmarks to be corrected are not limited to one, and it is considered that even though the correction for one landmark is completed, another landmark required to be corrected may be selected and corrected. Therefore, the display control unit 19 determines whether or not a new landmark has been selected after the correction of one landmark is completed (ST45), and when the new landmark is selected (YES in ST45), the display control unit 19 confirms the completion of the correction processing described above.

On the other hand, when the correction for the newly selected landmark is completed and it is determined that another landmark has not been selected (NO in ST45), the analysis-and-diagnosis unit 18 then determines whether or not all the corrections for the examination results that are currently listed are completed in all the times of measurement (ST46).

If the doctor performs processing such as selecting the analysis result of another time of measurement, the display control unit 19 determines that the landmark in the examination results of all the times of measurement has not been corrected (NO in ST46), the processing returns to Step ST37 (see FIG. 8), and a list of the examination results of all the times of measurement is displayed again. Then, the display control unit 19 accepts the correction processing of the landmark by the doctor described above.

On the other hand, when the display control unit 19 determines that the doctor does not continue to execute the correction processing (YES in ST46), the display control unit 19 displays, on the display 12, a list of images of all the times of measurement including the corrected times of measurement (ST47). As a result, the processing of analysis and diagnosis is terminated.

Moreover, as described above, the analysis-and-diagnosis unit 18 is also able to display, on the display 12, the calculated analysis result, the diagnosis result, and the reason for deriving the diagnosis result, together with the list.

When the doctor does not question the analysis and diagnosis of the examination result, the correction of the landmark as described above is not executed (NO in ST38 in FIG. 8), and the analysis-and-diagnosis processing is terminated at the point in time.

According to at least one embodiment described above, it is possible to automatically set the landmark required for analyzing the examination result for diagnosis of the esophageal motility disorder, and also to list images for each time of measurement when the positions of the landmarks that are automatically set after the analysis are confirmed.

Such landmarks are corrected, such that it is possible to ensure consistency between the analysis result or diagnosis result presented based on the examination result of the subject and the diagnosis result based on the experience and so on of the doctor.

Therefore, the diagnosis support apparatus 1 and the diagnosis support program according to the embodiment of the present invention are used, such that it is possible to perform a simpler and more reliable diagnosis.

While certain embodiments of the present invention have been described, the embodiments have been presented by way of example only, and are not intended to limit the scope of the invention. The embodiments described herein may be embodied in a variety of other forms, and furthermore, various omissions, substitutions and changes in the form of the embodiments described herein may be made without departing from the spirit of the invention. The accompanying claims and their equivalents are intended to cover such forms or modifications as would fall within the scope and spirit of the invention.

The following is a list of reference signs used in the drawings and the description.
1 diagnosis support apparatus
11 input unit
12 display
13 storage unit
14 communication control unit
15 removable disk
16 examination unit
17 landmark setting unit
18 analysis-and-diagnosis unit
19 display control unit
20 control unit

1. A diagnosis support apparatus comprising:
an examination unit configured to acquire a pressure when an object passes through a gastrointestinal tract inside a subject from a sensor placed along a longitudinal direction of the gastrointestinal tract;
a landmark setting unit configured to automatically set a landmark according to a predetermined condition based on an examination result acquired by the examination unit;
an analysis-and-diagnosis unit configured to analyze the examination result using the set landmark to acquire an analysis result; and
a display control unit configured to display the analysis result on a display unit.

2. The diagnosis support apparatus according to claim 1,
wherein when a doctor checks the landmark and needs to correct the landmark, the display control unit displays, on the display unit, a list of images showing the examination result for each time of measurement in the examination.

3. The diagnosis support apparatus according to claim 2,
wherein the display control unit displays, on the display unit, the image of a specific time of measurement selected by the doctor as a need for correcting the landmark together with the landmark.

4. The diagnosis support apparatus according to claim 2,
wherein the analysis-and-diagnosis unit analyzes the examination result again using a newly set landmark after the doctor has completed the correction of the landmark.

5. The diagnosis support apparatus according to claim 1,
wherein the analysis-and-diagnosis unit calculates a diagnosis result based on the analysis result, and the display control unit displays the diagnosis result on the display unit.

6. The diagnosis support apparatus according to claim 5,
wherein the display control unit also displays the diagnosis result together with a reason for calculating the diagnosis result on the display unit.

7. The diagnosis support apparatus according to claim 2,
wherein the analysis-and-diagnosis unit calculates the diagnosis result again based on the analysis result acquired using the newly set landmark when the doctor has completed the correction of the landmark.

8. A diagnosis support program causing a diagnosis support apparatus to execute a process comprising:
a step of acquiring an examination result for an examination that measures a pressure when an object passes through a gastrointestinal tract inside a subject;
a step of receiving an analysis request from a doctor and starting analysis processing for the examination result;
a step of automatically setting a landmark in each of a plurality of times of measurement constituting the examination result; and
a step of analyzing the examination result using the set landmark and acquiring an analysis result.

9. The diagnosis support program according to claim 8,
wherein the process further comprises:
a step of receiving a correction request of the landmark by the doctor after the analysis result of the examination result is displayed and displaying a list of images showing the examination result for each time of measurement.

10. The diagnosis support program according to claim 8,
wherein the process further comprises:
a step of calculating a diagnosis result based on the acquired analysis result, after the step of analyzing the examination result.

11. The diagnosis support apparatus according to claim 3,
wherein the analysis-and-diagnosis unit analyzes the examination result again using a newly set landmark after the doctor has completed the correction of the landmark.

12. The diagnosis support apparatus according to claim 3,
wherein the analysis-and-diagnosis unit calculates the diagnosis result again based on the analysis result acquired using the newly set landmark when the doctor has completed the correction of the landmark.

13. The diagnosis support apparatus according to claim 4,
wherein the analysis-and-diagnosis unit calculates the diagnosis result again based on the analysis result acquired using the newly set landmark when the doctor has completed the correction of the landmark.

14. The diagnosis support apparatus according to claim 5,
wherein the analysis-and-diagnosis unit calculates the diagnosis result again based on the analysis result acquired using the newly set landmark when the doctor has completed the correction of the landmark.

15. The diagnosis support apparatus according to claim 6,
wherein the analysis-and-diagnosis unit calculates the diagnosis result again based on the analysis result acquired using the newly set landmark when the doctor has completed the correction of the landmark.

16. The diagnosis support apparatus according to claim 11,
wherein the analysis-and-diagnosis unit calculates the diagnosis result again based on the analysis result acquired using the newly set landmark when the doctor has completed the correction of the landmark.
